(19) United States
(12) Patent Application Publication
(10) Pub. No.: US 2004/0124265 A1
(43) Pub. Date: Jul. 1, 2004

(54) Device and method for producing an aerosol

(76) Inventor: German Kaelberer, Owen/Teck (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/466,764

(22) PCT Filed: Jan. 31, 2002
(86) PCT No.: PCT/EP02/01004

(30) Foreign Application Priority Data
Jan. 31, 2001 (DE) 101 04 012.1

(51) Int. Cl. A61M 11/06

(52) U.S. Cl. 239/338

(57) ABSTRACT

The present invention relates to an aerosol-producing device (1) comprising an injector device (5) in which an aerosol (2) is produced from a carrier gas and a liquid. The carrier gas and the liquid are each supplied in controlled quantities to the injector device (5) via at least one throttle (8, 9) of a throttle system.

The present invention relates to an aerosol-producing device according to the preamble of claim 1.

Furthermore, the present invention relates to an aerosol-producing method.

DE 197 216 50 A1 discloses an aerosol producer in which flow control valves are provided for separately adjusting the amount of the carrier gas and/or the amount of the liquid that are each supplied to the injector devices.

The minimum quantity lubricator of DE 199 172 19 A1 discloses a metering member with two metering pumps the conveying capacity of which can be regulated through adjusting operations. Oil is conveyed by the two metering pumps from a supply vessel. Moreover, a needle valve is provided in the compressed-air supply line as part of the metering device.

DE 196 153 79 describes a pneumatic hand-held spray gun with automatic spray jet adjustment for use, for instance, in paint shops. External supply means for supplying at least horn air and/or medium to be applied have arranged therein devices which can be controlled by a control unit and via which the amount of application can be varied.

DE 92 05 369.3 discloses a device for the metered spraying, particularly, of adhesive. In this device, the liquid is aspirated by compressed air in a Venturi nozzle from a supply tank, whereby the amount of adhesive being adjustable in an infinitely variable way.

In the device of WO 98/10217, the amount of cooling lubricant which is aspirated for the atomization of the cooling lubricant is controlled by the help of a valve. The volume flow of the liquid cooling lubricant supplied for atomization and the volume flow of the compressed air are here variable via a pressure reducing valve.

A further atomizer is known from EP 0 941 769 A1.

Finally, the post-published EP 1 106 902 A1 describes an atomizer in which only the tank pressure, but not the amount of liquid and/or carrier gas, can be controlled in dependence on the supply pressure.

A generic device is known from DE 196 543 21 A1 . The aerosols produced thereby can be used in inhalators in the medical field, air humidifiers for domestic applications, and especially for cooling and lubricating tools or workpieces.

The device comprises an injector device arranged in a pressure tank for producing an aerosol from a liquid and a carrier gas. The liquid forming a lubricant, preferably oil, is aspirated by means of a vacuum or low pressure and atomized in a jet of the carrier gas, preferably air. The carrier gas is introduced under pressure into a chamber of the injector device where due to the enlarged cross-section a vacuum is obtained that conveys the liquid from its conduit also terminating in the chamber and supplies it at a high velocity to the carrier gas flow. The liquid is entrained with the carrier gas flow and deposits finely distributed on the structured surface of an impact body. The liquid film is atomized into an aerosol of a small particle size on the surface of the impact body.

In the lid of the pressure tank a connection conduit is provided which forms an aerosol outlet and via which aerosol is taken from the pressure tank to supply it at least to a tool or workpiece for cooling and lubrication.

The liquid and the carrier gas are each supplied to the injector device via a conduit in which a control valve is provided. The control valves regulate the pressure, for the supply of the liquid as well as for the supply of the carrier gas.

The device designed in this way ensures a continuous aerosol production as long as a sufficiently large amount of aerosol is conveyed via the aerosol outlet from the pressure tank. This is the case when the tools connected to the aerosol outlet have at least a minimum diameter typically of 10 mm.

With smaller tool diameters the withdrawal amount of the aerosol per time unit is so small that the tank pressure inside the pressure tank rises continuously, whereby the tank pressure asymptotically approaching the supply pressure at which the carrier gas is supplied via an external carrier gas supply to the injector device.

This rise in pressure can not be prevented through the pressure control on the control valves. To be more specific, a pressure control can no longer be carried out via the control valve for the carrier gas supply because the supply pressure for the carrier gas is fixedly predetermined in accordance with the configuration of the external carrier gas supply.

Thus, independently of the pressure control by the control valve for the liquid supply, the differential pressure between supply pressure and tank pressure becomes so small that the flow velocity of the carrier gas supplied to the injector device is no longer high enough for aspirating liquid. Hence, only carrier gas without liquid is supplied to the injector device, so that the aerosol production is interrupted.

To maintain the aerosol production in such cases, a bypass is installed in the pressure tanks of known devices. Aerosol is discharged via the bypass in the case of an inadmissible rise in pressure in the pressure tank, whereby the tank pressure decreases again.

Apart from the fact that the aerosol discharged from the pressure tank is no longer available for tool lubrication and the yield of aerosol production decreases in an undesired way, such a bypass requires downstream devices to collect and dispose of the discharged aerosol. As a result, the design efforts taken for the aerosol-producing device are intensified in an undesired way.

It is the object of the invention to design a device of the above-mentioned type such that a reliable aerosol production is ensured for a range of aerosol withdrawal amounts that is as wide as possible.

To achieve said object, the features of claim 1 are provided. Advantageous embodiments and expedient developments of the invention and a method which also achieves the above object are described in the further claims.

The aerosol-producing device according to the invention comprises an injector device. In the injector device, an aerosol is produced from a carrier gas and a liquid. The carrier gas and the liquid are each supplied in controlled quantities to the injector device via at least one throttle of a throttle system.

The quantity-controlled supply of carrier gas and liquid according to the invention into the injector device results in an equally reliable aerosol production both with small and large aerosol withdrawal amounts. It is particularly advantageous that within the whole range of aerosol withdrawal amounts the proportion of carrier gas and liquid in the aerosol can be predetermined in a selective and reproducible way.

This advantage is mainly due to the fact that the quantity-controlled supply of carrier gas and liquid into the injector device is optimally adapted to the aerosol withdrawal process.

The units supplied with aerosol, particularly tools, are connected to the device via at least one aerosol outlet. The connection of a tool to an aerosol outlet has an effective aerosol withdrawal cross-section via which, depending on the design and mode of operation of the tool, a specific volume flow of aerosol is supplied. The tool itself thereby forms a throttle via which a specific volume flow of aerosol is conveyed.

The basic idea of the invention is that the volume flows of the carrier gas and the liquid that are introduced into the injector device are adapted to the aerosol withdrawal amounts by means of quantity control via the throttle system. Neither the pressure of the carrier gas nor the pressure of the liquid is here regulated. Rather, a dynamic pressure change in the carrier gas supply and the liquid supply takes place on the throttle system.

Said dynamic change in pressure has a stabilizing effect on aerosol production and on the supply of the connected tool. Especially a self-stabilization of the aerosol production and aerosol supply takes place in the case of time-variable aerosol withdrawal amounts.

For instance, when a drill to be cooled and lubricated is connected to the aerosol outlet, a constant amount of aerosol will be conveyed via the aerosol outlet as long as the operative state thereof remains unchanged. Accordingly, specific volume flows of carrier gas and liquid are conveyed via the throttle system. When the drill changes its operative state, e.g. by the drill penetrating into a workpiece, the aerosol withdrawal cross-section will be reduced so that a smaller amount of aerosol is supplied to the tool. Since the supply of liquid and carrier gas to the injector device is not pressure-controlled, but quantity-controlled, the aerosol pressure in front of the aerosol outlet is increased, whereby a larger volume flow is again supplied to the tool, which in turn leads to pressure stabilization in the interior of the device for aerosol production.

In an advantageous embodiment, the quantities are controlled in response to a monitoring of the differential pressure with a sensor which measures the pressure difference ΔP between the supply pressure Pᵥ, with which the carrier gas is supplied to the injector device, and the tank pressure P_{B} in the pressure tank in which the injector device is arranged.

When the differential pressure ΔP is above a first limit value ΔP₀, corresponding volume flows of carrier gas and liquid are supplied to the injector device with a given adjustment of the throttle system. When the differential pressure ΔP decreases, so that it is below ΔP₀, the throttle system will be closed and aerosol production suppressed. Aerosol production will only be resumed again when the differential pressure exceeds a further limit value ΔP₁ which is above ΔP₀. The difference between ΔP₁ and ΔP₀ forms a switching hysteresis. This avoids unnecessary switching operations, resulting in a stabilization of the aerosol production.

Such a control yields an aerosol production showing a long-term stability, both for very small and very large aerosol withdrawal amounts. Accordingly, tools having large and also tools having small aerosol withdrawal cross-sections can be connected to the aerosol outlets. Especially tools having tool diameters of less than 1.5 mm, which are e.g. used in deep-hole drilling, can be reliably supplied with aerosol. Thereby it is especially impossible that pressure conditions are prevailing in the pressure tank that eliminate an aerosol production.

Furthermore, it is particularly advantageous that the volume flows of the carrier gas and the liquid can be predetermined via a fixedly predetermined setting of the throttle system and need not to be changed during operation of the device.

In an advantageous embodiment of the invention, the control of the device and the setting of the throttle system, in particular, are performed centrally via a control unit.

In one embodiment the throttle system comprises baffle assemblies controlled by the control unit, each having a predetermined number of baffle openings that can be opened or closed selectively via the control unit. This kind of setting the volume flow with the help of baffles represents a separate advantageous aspect that is independent of the remaining design of the apparatus.

At least one nozzle with a baffle assembly is provided for the supply of carrier gas and at least one further nozzle with a further baffle assembly for the supply of liquid.

A further essential advantage of the device of the invention is that aerosol can be withdrawn with this device at predetermined time intervals without the device assuming an unstable state.

Such modes of operation are e.g. required for cooling and lubricating drills which have to be fed with aerosol at predetermined time intervals. For this a closing means, which is e.g. formed by a controlled ball valve, is arranged at the aerosol outlet. The aerosol withdrawal can be started or finished abruptly by a corresponding control of the ball valve.

Due to this interval operation the pressure conditions vary in the pressure tank. On account of the control times that are due to the system, a pressure-controlled supply of carrier gas and liquid would be too inert to follow these changes. By contrast, in the device of the invention, there is no pressure control whatsoever so that even by closing of the ball valve the aerosol will be produced further with unchanged parameters of the throttle system. The aerosol produced as a reserve can thus exit suddenly by opening of the ball valve and is thus directly available to the tool.

The invention will now be explained hereinafter with reference to the drawings, in which:

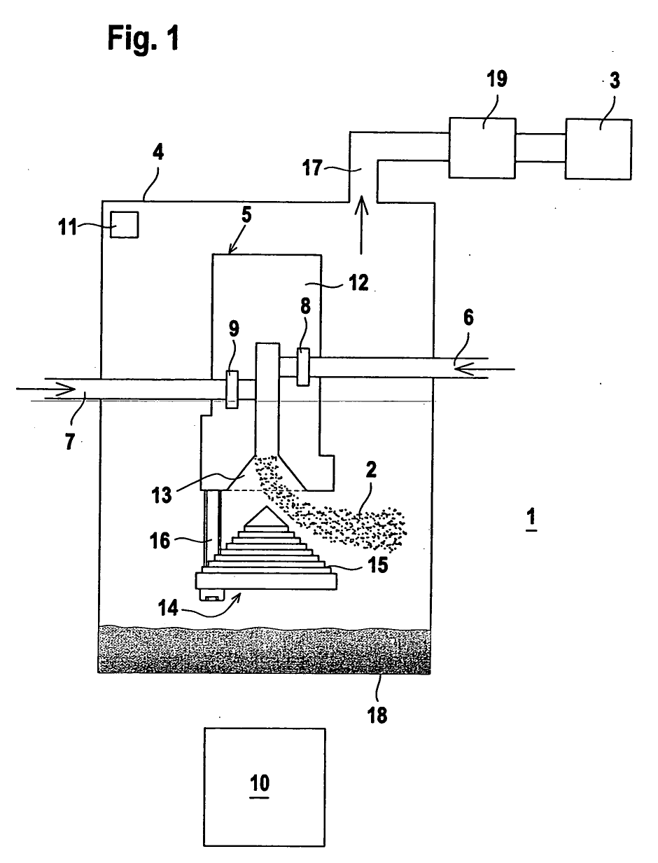

FIG. 1 is a block diagram of an embodiment of the aerosol-producing device according to the invention.

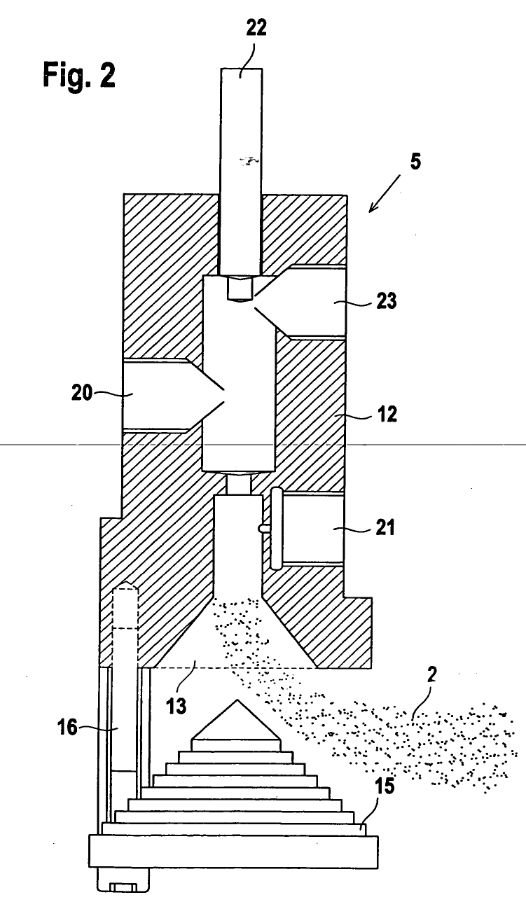

FIG. 2 shows a detail of a section of a second embodiment of an aerosol-producing device.

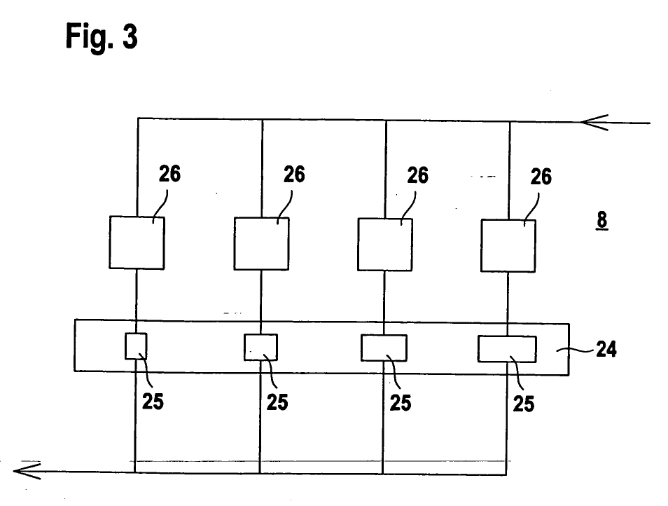

FIG. 3 shows an embodiment of a baffle assembly for the device according to FIG. 1.

FIG. 1 is a schematic view showing the basic structure of an aerosol-producing device 1. In the present example, the aerosol 2 produced by means of the device 1 is used for cooling and lubricating tools 3, workpieces, and also gears and machines. In principle, such devices 1 can also be used for producing aerosols 2 for inhalators in the medical field, air humidifiers for domestic applications, or the like.

The device according to FIG. 1 comprises a pressure tank 4 in which an injector device 5 is arranged. The injector device 5 is fed with carrier gas via a first conduit 6 and with a liquid via a second conduit 7. The carrier gas consists of air and is made available via a compressed-air supply (not shown) at a supply pressure Pᵥ, which is typically in the range of 6 bar≦Pᵥ≦10 bar. The liquid serves as a lubricant and is formed in the present embodiment by oil which is fed via an oil reservoir into conduit 7. Alternatively, synthetic ester, or the like, may also be used.

Both air and oil are each supplied via a throttle 8, 9 to the injector device 5. The throttles 8, 9 forming a throttle system are controlled via a control unit 10. The control unit 10 is formed by an electric circuit, or the like. Next to throttles 8, 9, a sensor 11 is connected to the control unit 10 via leads (not shown). The sensor 11, which is designed as a differential pressure sensor, measures the differential pressure ΔP=Pᵥ−P_{B}, where P_{B} is the pressure prevailing in the interior of the pressure tank 4.

The injector device 5 comprises an injector block 12 having arranged therein an injector chamber 13. The injector chamber 13 is enlarged in the area of its outlet towards the lower end. A conical impact body 14 is arranged below the outlet. The outer surface of said impact body has a stepped structure including a multitude of successive steps 15.

The impact body 14 is arranged via a mounting 16 at a predetermined distance relative to the outlet of the injector chamber 13, said distance being preferably adjustable.

Air and oil are supplied via the throttles 8, 9 into the injector chamber 13, and a vacuum by which oil is aspirated and intermixed with the compressed air is created by entry into the injector chamber 13 due to the enlarged cross-section. The resulting gas jet with the liquid droplets contained therein is supplied to the impact body 14 on which liquid droplets are deposited first. The succeeding high-velocity air flow of the gas jet including further liquid droplets will lead, by impact of said droplets, to the formation of particulate droplets of the oil, resulting in the formation of an aerosol 2 with very fine oil particles.

The aerosol 2 with the fine oil particles is deflected laterally and discharged via an aerosol outlet 17, which is arranged in the instant example on the ceiling of the pressure tank 4, from said tank and supplied to a tool 3. In principle, a plurality of aerosol outlets 17 may also be provided for connection to a predetermined number of tools 3.

The heavy oil particles will descend onto the bottom of the pressure tank 4, in which an oil reservoir 18 is arranged that is advantageously used as oil for aerosol production.

To achieve a size selection of the oil droplets in the aerosol 2, cage structures (not shown) may be arranged in the area in front of the aerosol outlet 17 for enforcing an approximately rectangular deflection of the aerosol stream. Large oil drops cannot follow said deflection, so that only smallest oil droplets in the aerosol stream are guided via the aerosol outlet 17 out of the pressure tank 4.

In addition, a further nozzle (not shown) may be provided for supplying additional air.

A closing means 19 which is controlled by an external control is provided between aerosol outlet 17 and tool 3. The closing means 19 is preferably formed by a ball valve which is controlled by compressed air. Said valve can be closed and opened via the control with short response times, whereby the supply of aerosol 2 to the tool 3 can be activated and deactivated abruptly.

FIG. 2 shows an embodiment of an injector device 5, which is substantially identical with the embodiment according to FIG. 1. The injector device 5, in particular, also comprises an injector chamber 13 which is opposite to and spaced apart from the impact body 14, the impact body 14 being fixed by means of the mounting 16 at a predetermined distance relative to the injector chamber 13.

In contrast to the embodiment according to FIG. 1, two sets of nozzles with two nozzles 20, 21, 22, 23 each are provided in the arrangement according to FIG. 2, to feed air and oil into the injector device 5.

The first set of nozzles comprises two nozzles 20, 21 closely adjoining the injector chamber 13, air being supplied via the first nozzle 20 and oil via the second nozzle 21 into the injector chamber 13.

The second set of nozzles is positioned above the first set of nozzles and also comprises a nozzle 22 for feeding air and a second nozzle 23 for feeding oil.

The supply of oil and air via the various nozzles is quantity-controlled and takes place via a respective throttle 8, 9 (not shown in FIG. 2), the throttles 8, 9 forming the throttle system.

The control of the throttle system and the supply of oil and air into the injector device 5 again takes place via the control unit 10.

Depending on the amount of oil required for aerosol production, oil and air are introduced into the injector device 5 preferably either only via the first nozzle system or via both nozzle systems. The air supply can be further adjusted by means of the additional nozzle 22.

The throttles 8, 9 of the throttle system are designed in the form of baffle assemblies. An embodiment of such a throttle 8 designed as a baffle assembly for the supply of air is illustrated in FIG. 3. Such a baffle assembly can be used in the aerosol producer independently of the use of a differential pressure control.

The baffle assembly comprises a baffle body 24 having incorporated thereinto a predetermined number of baffle openings 25 spaced apart side by side. Each of the baffle openings 25 has a circular cross-section. The diameters of the baffle openings 25 are preferably designed to differ from one another and are within the range of less than one millimeter up to a few millimeters. The baffle assembly shown in FIG. 3 serves to supply air and comprises four adjacent baffle openings 25. A valve 26 via which air is supplied to the respective baffle opening 25 is positioned in front of each baffle opening 25.

The diameters of the baffle openings 25 are large in comparison with the thickness of the baffle body 24. At any rate, the diameters are at least a third of the thickness of the baffle body. As a consequence, the longitudinal extension of the baffle opening 25 is considerably smaller, but at least not substantially larger than the cross section thereof. This has the effect that even at different flow velocities of the air across the baffle cross-section, a substantially constant and homogeneous velocity profile is obtained.

The baffle openings 25 of each baffle assembly can be closed or opened selectively via the control unit 10. Depending on which ones of the baffle openings 25 are closed or opened, different cross-sections of the throttles 8, 9 are obtained, whereby fifteen different cross-sections being adjustable in the present case.

The baffle assemblies for the supply of oil have a structure which is substantially identical with the structure of the baffle assembly according to FIG. 3. The baffle assembly for the supply of oil only comprises two different baffle openings 25 in the present case.

The baffle assembly for the additional air comprises three baffle openings 25 with different cross-sections in the present case.

The aerosol-producing device 1 is centrally controlled via the control unit 10.

Depending on the type of the tool 3 connected to the aerosol outlet 17, it is predetermined for the individual baffle assemblies via the control unit 10 which ones of the baffle openings 25 are closed and which ones are opened. Depending on the aerosol withdrawal amount discharged via the aerosol outlet 17 out of the pressure tank 4, there will be consequently an adjustment of the oil and air volume flows supplied to the injector device 5. By the specification of said volume flows, a dynamic pressure control takes place in the interior of the pressure tank 4, which particularly results in a self-stabilization of the aerosol production. Dynamic cross-sectional changes in the cross-section of the aerosol discharge, in particular, as well as associated changes in the aerosol withdrawal amounts, effect a change in pressure in the interior of the pressure tank 4, which counteracts the respective change in the withdrawal amount. Such dynamic processes are particularly observed in tools 3 formed as drills, which are introduced into a workpiece and then removed from the workpiece again.

This yields a stable aerosol production without the need for changing the setting of the throttle system during operation of the device 1. A stable aerosol production, in particular, is ensured in the case of time-variable aerosol withdrawal amounts.

The set values of the throttle system are suitably stored as parameter values or characteristic curves in the control unit 10 or are entered into the unit, if necessary.

The largely sole precondition for a stable aerosol production is that the differential pressure ΔP=Pᵥ−P_{B} should be above a predetermined limit value ΔP₀. At a predetermined supply pressure Pᵥ on the compressed-air supply means, the tank pressure P_{B} in the pressure tank 4 must then not exceed a predetermined limit value. The limit value ΔP₀ is in the range of 2 bar<P₀<2.5 bar and is preferably 2.2 bar. The differential pressure is sensed by means of the sensor 11 and is read as an aerosol production control input into the control unit 10.

When the differential pressure is above the limit value ΔP₀, a continuous aerosol production takes place in the injector device 5 with the settings of the baffle assemblies that are predetermined in the control unit 10.

When the differential pressure ΔP is below the limit value ΔP₀, the aerosol production is suppressed. Preferably, all baffle assemblies are closed for this purpose. The aerosol outlet 17, however, remains open, so that aerosol 2 is still conveyed out of the pressure tank 4, whereby the tank pressure P_{B} decreases in the course of time.

Aerosol production will only be resumed again if the differential pressure ΔP exceeds a further limit value ΔP₁. The limit value ΔP₁ is above the limit value ΔP₀. Preferably, ΔP₁ is in the range of 3 bar≦ΔP₁≦3.5 bar and is 3.4 bar in a particularly advantageous way.

The difference ΔP between the limit values ΔP₀ and ΔP₁ forms a switching hysteresis for the aerosol production control, whereby unnecessary switching operations, i.e. start and interruption of the aerosol production, are avoided. This prevents an escalating of the system.

Especially with tools 3 designed as drills, it is necessary that aerosol 2 should be supplied to the respective tool 3 only at fixedly predetermined time intervals.

For example, it is necessary to limit the aerosol supply to the time intervals during which the drill is inserted in a workpiece. By contrast, aerosol production should be stopped when tool 3 is removed from the workpiece in order to avoid unnecessary soiling of the tool with oil.

To this end, the ball valve is operated via an external control, whereby the aerosol supply to the tool 3 can be abruptly activated and deactivated.

1. An aerosol-producing device comprising an injector device (5) which is arranged in a pressure tank (4) subjected to a tank pressure (P_{B}), and in which an aerosol is produced from a carrier gas supplied at a supply pressure (Pᵥ) and from a liquid, and comprising at least one throttle (8, 9) via which the carrier gas and the liquid are each supplied in controlled quantities to the injector device, characterized in that the throttle is designed to be controllable in dependence upon the differential pressure (ΔP=Pᵥ−P_{B}) between tank pressure and supply pressure.

2. The aerosol-producing device according to claim 1, characterized in that said throttle (8, 9) is controlled via a control unit (10).

3. The aerosol-producing device according to claim 1 or 2, characterized in that a sensor (11) is provided for determining the differential pressure (ΔP=Pᵥ−P_{B}) which forms an input for said control unit (10).

4. The aerosol-producing device according to any one of the aforementioned claims, characterized in that said at least one throttle (8, 9) is part of a throttle system which comprises a baffle assembly including a plurality of throttles designed as baffle openings (25).

5. The aerosol-producing device according to any one of the aforementioned claims, characterized in that said carrier gas is supplied via a baffle assembly forming at least one nozzle (20) and a first throttle (8), and said liquid is supplied via a baffle assembly forming at least one further nozzle (21) and a further throttle (9), to said injector device (5).

6. The aerosol-producing device according to any one of the aforementioned claims, characterized in that two sets of nozzles with two nozzles (20, 21, 22, 23) each are provided for the supply of carrier gas and liquid, each nozzle (20, 21, 22, 23) having a separate baffle assembly assigned thereto.

7. The aerosol-producing device according to claim 6, characterized in that a nozzle (22) with an upstream baffle assembly is provided for the supply of additional carrier gas.

8. The aerosol-producing device according to any one of claims 4 to 7, characterized in that each baffle assembly has a predetermined number (N) of separately closable baffle openings (25).

9. The aerosol-producing device according to any one of claims 4 to 8, characterized in that said baffle openings (25) of one baffle assembly are each equipped with different diameters.

10. The aerosol-producing device according to any one of claims 4 to 9, characterized in that the diameter of each baffle opening (25) of a baffle assembly is at least one third of the thickness of the baffle body (24) defining said baffle opening (25).

11. The aerosol-producing device according to any one of claims 4 to 10, characterized in that each baffle opening (25) of a baffle assembly has carrier gas or liquid supplied thereto via a separate valve (26).

12. The aerosol-producing device according to any one of the aforementioned claims, characterized in that the aerosol outlet (17) has assigned thereto a controllable closing means (19) by the actuation of which the supply of aerosol (2) via said aerosol outlet (17) is interrupted.

13. The aerosol-producing device according to claim 11, characterized in that said closing means (19) is formed by a controlled ball valve.

14. The aerosol-producing device according to any one of the aforementioned claims, characterized in that said carrier gas is formed by air, and that said liquid is formed by oil or a synthetic ester.

15. The aerosol-producing device, especially according to any one of the aforementioned claims, comprising an injector device in which an aerosol is produced from a carrier gas and a liquid, said carrier gas and said liquid being supplied in controlled quantities to said injector device via a respective throttle system, characterized in that said throttle system comprises a baffle assembly with separately closable baffle openings.

16. A method for producing an aerosol, wherein in an injector device (3) in a pressure tank (4) subjected to a tank pressure (P_{B}), an aerosol (2) is produced from a carrier gas supplied at a supply pressure (Pᵥ) and from a liquid, characterized in that the volume flow of said carrier gas and said liquid is controlled in dependence upon the pressure difference (ΔP=Pᵥ−P_{B}) between said tank pressure and said supply pressure.

17. The method for producing an aerosol according to claim 16, characterized in that baffle openings (25) are opened or closed individually via said control unit (10).

18. The method for producing an aerosol according to claim 16 or 17, characterized in that said baffle openings (25) of said baffle assemblies are closed by said control unit (10) as soon as said differential pressure (ΔP) has fallen below a first predetermined limit value (ΔP₀).

19. The method for producing an aerosol according to claim 18, characterized in that said first limit value (ΔP₀) is within a range between 2 bar and 2.5 bar.

20. The method for producing an aerosol according to any one of claims 17 to 19, characterized in that predetermined baffle openings (25) of said baffle assemblies are opened by said control unit (10) as soon as said differential pressure (ΔP) has exceeded a second predetermined limit value (ΔP₁), said second predetermined limit value (ΔP₁) being greater than said first predetermined limit value (ΔP₀).

21. The method for producing an aerosol according to claim 20, characterized in that said second limit value (ΔP₁) is within a range between 3 bar and 3.5 bar.

22. The method for producing an aerosol according to any one of claims 16 to 20, characterized in that the number of the opened baffle openings (25) of the individual baffle assemblies is predetermined via said control unit (10) at a differential pressure above said first limit value (ΔP₀) in dependence upon the quantity of aerosol (2) conveyed via at least one aerosol outlet (18) out of said pressure tank (4).

23. The method for producing an aerosol according to claim 20, characterized in that said baffle openings (25) of said individual baffle assemblies are opened in dependence upon parameter values and/or characteristic curves.

24. The method for producing an aerosol according to claim 23, characterized in that the parameter values and/or characteristic curves are entered into and/or stored in said control unit (10).

25. The method for producing an aerosol according to any one of claims 16 to 24, characterized in that said aerosol (2) is used for the cooling lubrication of tools (3), workpieces and/or tool parts.

26. The method for producing an aerosol according to any one of claims 16 to 25, characterized in that said supply pressure (Pᵥ) is in the range between 6 bar and 10 bar.

27. The method for producing an aerosol, especially according to any one of the above-mentioned claims, wherein in an injector device an aerosol is produced from a carrier gas and a liquid, the quantity of the carrier gas supplied into said injector device and that of the liquid supplied into said injector device being controlled, characterized in that a plurality of baffle openings are selectively opened and closed for quantity control.
